(19) United States
(12) Patent Application Publication
(10) Pub. No.: US 2004/0029204 A1
(43) Pub. Date: Feb. 12, 2004

(54) Method for obtaining human cdc25 phosphatases and method for identifying human cdc25 phosphatase modulators

(76) Inventors: Francoise Goubin-Gramatica, Agen (FR); Bernard Ducommun, Belberaud (FR); Gregoire Prevost, Antony (FR)

Correspondence Address: Bierman Muserlian & Lucas, 600 Third Avenue, New York, NY, 10016, US

(21) Appl. No.: 10/149,472

(22) PCT Filed: Dec. 13, 2000
(86) PCT No.: PCT/FR00/03496

(30) Foreign Application Priority Data
Dec. 14, 1999 (FR) 99/15722

(51) Int. Cl. C12Q 1/34; C07H 21/04; C12N 9/24; C12P 21/02; C12N 1/21; C12N 15/74

(52) U.S. Cl. 435/18; 435/69.1; 435/200; 435/252.3; 435/320.1; 536/23.2

(57) ABSTRACT

The invention concerns a method for obtaining human cdc 25B1, cdc25B2, cdcB3 and cdc25C phosphatases. More particularly, the invention concerns a protein for fusion between human cdc25B1, cdc25B2, cdcB3 or cdc25C phosphatase and the maltose binding protein (MBP) of Escherichia coli, the DNA sequence coding for said fusion protein, a method for preparing said fusion protein and a method for identifying human cdc25B1, cdc25B2, cdcB3 or cdc25C protein modulators.

The present invention relates to a method for obtaining human Cdc25 phosphatases. It also relates to a method for identifying human Cdc25 phosphatase modulators.

Entry of the cell into the cell division process is regulated by a group of kinases and phosphatases useful for the synchronisation of the different phases of the cell cycle and allow reorganisation of cell architecture.

Cycline dependent kinases (CDKs) play a major role in this control and already several inhibitors of this kinase family have been identified. One of these compounds (flavopiridol) is already in clinical Phase II (Senderowicz and Sausville, J. Natl. Cancer Inst. (2000), 92, 376-387).

These CDKs are activated by dephosphorylation carried out by the Cdc25 phosphatases, on tyrosine and threonine residues. In human cells, the Cdc25 proteins are coded by one family: Cdc25A, Cdc25B and Cdc25C (Cans et al., Medicine Sciences (1998), 3, 269-274).

A few Cdc25 inhibitors have been identified, but they have only a weak activity (cf. Baratte, B., Meijer, L., Galaktionov, K., and Beach, D., Anticancer Res. (1992), 12, 873-880; Rice, R. L. et al., Biochemistry (1997), 36, 15965-15974; and Ham, S. W., Park, J., Lee, S. J., Kim, W., Kang, K., and Choi, K. H., Bioorg. Med. Chem. Lett. (1998), 8, 2507-2510).

Cdc25B2 is a protein tyrosine phosphatase similar to Cdc25B phophatase (now called Cdc25B1). It was identified in a DNA bank coding for Burkitt's lymphoma. Cdc25B2 differs from Cdc25B1 by an insertion of 14 amino acids and a deletion of 41 amino acids upstream of the catalytic domain. Cdc25B1 and Cdc25B2 are splicing variants of the same gene. A third variant, Cdc25B3, carrying the two sequences of 14 and 41 amino acids, was identified from the same bank (Baldin et al., Oncogene (1997), 14, 2485-2495). The three variants are detected in the primocultures and the cell lines. Analysis of the variants shows that Cdc25B2 is more weakly expressed than Cdc25B3 in all of the lines tested but that the expression of the two variants increases during the G2 phase and mitosis (Forrest et al., Biochem. Biophys. Res. Commun. (1999), 260, 510-515). Hernandez et al. report that Cdc25A and -B2 but not Cdc25B1, -B3 and -C, are overexpressed in a large number of lymphomas (35% and 39%) (Hernandez et al., Int. J. Cancer (2000), 89(2), 148-52). The normal lymphocytes express the Cdc25B 1 and -B3 messengers and very weakly those of Cdc 25A, -B2 and -C.

The over-expression of the three Cdc25B variants in yeast shows that Cdc25B2 appears to be more active than B1 or B3 (B2>B3>B1). The alternative splicing of Cdc25B can therefore play a role in the control of cell proliferation.

Cdc25C phosphatase is moreover itself regulated by phosphorylation on serine-216 by other Cds1 or Chk1 enzymes and binds itself to highly conserved members of the 14-3-3 protein family (Zeng, Y. et al., Nature (1998), 395, 507-510).

The search for more effective phosphatase inhibitors must overcome the necessity of having a protein which retains its phosphatase activity, in a non-limited quantity so as to allow large scale screening.

The study of a protein requires large quantities of the latter to satisfy all of the characteristics which can be analysed, in particular in fields such as biophysics (size, sequence, structure etc.), biochemistry (activity, stability, regulation etc.) or pharmacology (activators, inhibitors, etc.).

The production and the purification of this protein in large quantities comes up against several obstacles which can be:

absent or too weak an expression of the protein;

expression of a truncated protein;

obtaining a protein without biological activity;

loss of the biological activity of the protein during purification;

very weak purification yield;

loss of the biological activity of the protein during storage;

loss of the production source.

Only successful completion of each of these stages finally allows the long term production of a biologically-active protein in unlimited quantities. Faced with such requirements, the purification of a number of proteins remains a very delicate even impossible process in some cases.

However, much effort has been made to develop new production and purification strategies such as:

large scale cell culture (animal or vegetable);

large scale microorganism culture (bacteria, yeasts);

immunoprecipitation from a mixture of proteins using a specific antibody;

affinity chromatography, using a specific ligand (effectors, repressors, activators);

bidimensional electrophoresis, as a function of the molecular weight and the isoelectric point of the protein;

capillary electrophoresis;

enrichment by differential precipitation with different salts;

etc.

Moreover, new systems now try to combine the production and the purification of proteins. These systems allow an often inducible expression of the production of a recombinant protein fused with a protein allowing affinity chromatography (called tag protein). This last part can be eliminated by the addition of a protease which specifically recognises the location of the fusion (Sheibani, N., Prep. Biochem. Biotechnol. (1999), 29, 77-90). The number of systems proposed increases but the success of these different approaches remains very varied depending on the proteins to be purified. The conformation and the solubility of the proteins remain parameters which are impossible to control in these new systems (see Guise, A. D., West, S. M. and Chaudhuri, J. B., Mol. Biotechnol. (1996), 6, 53-64; Kelley, R. F. and Winkler, M. E., Genet. Eng. (N.Y., 1990), 12, 1-19).

The Cdc25B 1, Cdc25B2, Cdc25B3 or Cdc25C proteins could be fused with systems as varied as:

1. The residue of six histidines recognised by six histidine anti-motif antibody (Katsafanas, G. C. and Moss, B., Virology (1999), 258, 469-479);

2. The residue of nine amino acids of hemaglutinin from influenza recognised by 3F10 antibodies (Robert, I. and Quirin-Stricker, C., J. Mol. Neurosci. (1998), 11, 243-251);

3. The residue of 11 amino acids of the vesicular stomatitis virus recognised by the P5D4 antibody (The Maout, S., et al., Proc. Natl. Acad. Sci. U.S.A. (1997), 94, 13329-13334);

4. The residue of 6 amino acids of the capside protein of bovin papilloma virus (AU1) recognised by the anti-AU1 antibody (The Maout, S., et al., Proc. Natl. Acad. Sci. U.S.A. (1997), 94, 13329-13334);

5. The residue of 12 amino acids of the heavy chain of the C protein recognised by the HPC4 antibody (Rezaic, A. R., et al., Protein Expr. Purif. (1992), 3, 453-460);

6. The C-myc protein recognised by the 9E10 antibody (Bae, S. H., et al., J. Biol. Chem. (1999), 274, 14624-14631);

7. The beta galactosidase protein affinity for amino-phenyl-β-D-thiogalactopyranoside (Germino, J. and Bastia, D., Proc. Natl. Acad. Sci. U.S.A. (1984), 81, 4692-4696);

8. The gluthation S-transferase protein recognised by the anti-GST antibody (Carr, S., et al., Vaccine (1999), 18, 153-159);

9. The biotin-carboxylase carrier has an affinity for avidine (Germino, F. J. and Moskowitz, N. K., Methods Enzymol. (1999), 303, 422-450);

10. The intein protein has an affinity for chitin (see Chong, S., et al., Gene (1997), 192, 271-281; Carr, S., et al., Vaccine (1999), 18, 153-159);

11. The maltose binding protein has an affinity for amylose (Ahaded, A., et al., Prep. Biochem. Biotechnol. (1999), 29, 163-176).

Even so, nothing makes it possible to predict whether the operation will actually be successful.

However, the applicant has just perfected a method which allows human Cdc25B1, Cdc25B2, Cdc25B3 and Cdc25C enzymes to be obtained in an active form and in unlimited quantities. The present invention on one hand facilitates the research and study of the physiological or/and physio-pathological actions of this protein and on the other hand facilitates research for agents modulating these activities.

The invention makes it possible to obtain a recombinant human Cdc25B1, Cdc25B2, Cdc25B3 or Cdc25C protein with the maltose binding protein (MBP) which retains its phosphatase activity without necessitating separation with the MBP part, thus preventing any contamination with the proteases. The high level of the expression rate of the protein after induction allows an excellent purification yield and preparation of the enzyme in unlimited quantities.

Therefore a subject of the invention is firstly a fusion protein between the maltose binding protein (MBP) and a protein chosen from the Cdc25B1, Cdc25B2, Cdc25B3 and Cdc25C proteins.

The present invention in particular relates to a protein chosen from the following proteins:

a fusion protein between human Cdc25B 1 phophatase and the MBP, which is characterized in that it is coded by the sequence SEQ. ID No. 12 (represented further on);

a fusion protein between human Cdc25B2 phophatase and the MBP, which is characterized in that it is coded by the sequence SEQ. ID No. 13 (represented further on);

a fusion protein between human Cdc25B3 phophatase and the MBP, which is characterized in that it is coded by the sequence SEQ. ID No. 14 (represented further on); or

a fusion protein between human Cdc25C phosphatase and the MBP, which is characterized in that it is coded by the sequence SEQ. ID No. 1 (represented further on).

A subject of the invention is also the DNA coding for the said fusion proteins, as well as the DNA complementary to the DNA coding for the said fusion protein.

In addition a subject of the invention is the bacterial strain JM 109 transfected:

by the plasmid with sequence SEQ. ID No. 9 (represented further on), said strain being useful in the preparation of the Cdc25B1 protein;

by the plasmid with sequence SEQ. ID No. 10 (represented further on), said strain being useful in the preparation of the Cdc25B2 protein;

by the plasmid with sequence SEQ. ID No. 11 (represented further on), said strain being useful in the preparation of the Cdc25B3 protein; or

by the plasmid with sequence SEQ. ID No. 5 (represented further on), said strain being useful in the preparation of the Cdc25C protein.

In addition, the invention relates to a process for the preparation of the said fusion proteins, characterized in that it comprises the following successive stages:

culture of the bacterial strain JM 109 transfected by the plasmid with sequence SEQ. ID No. 9, the plasmid of sequence SEQ. ID No. 10, the plasmid with sequence SEQ. ID No. 11 or the plasmid with sequence SEQ. ID No. 5, in an LB medium with added ampicillin;

induction of the fusion protein synthesis by adding isopropylthiogalactoside;

lysis of the bacteria;

purification of the fusion protein obtained by chromatography on amylose-agarose resin and recovery of the fractions containing the purified protein.

Finally the invention relates to a use of said fusion protein in a method for identifying modulators of the Cdc25B1, Cdc25B2, Cdc25B3 or Cdc25C protein, characterized in that it comprises the following successive stages:

addition, of the fusion protein as obtained by the preparation process described previously and a compound presumed to be a modulator of the Cdc25B1, Cdc25B2, Cdc25B3 or Cdc25C protein to a solution of 3-O-methylfluorescein phosphate;

determination of the quantity of 3-O-methylfluorescein produced in relation to the initial quantity of 3-O-methylfluorescein phosphate.

Determination of the quantity of 3-O-methylfluorescein produced can be carried out, for example, by measuring the optical density of the solution, of the absorbance linked to 3-O-methylfluorescein at the wavelength of 477 nm, or also by fluorometry using excitation at the wavelength of 475 nm and reading at the wavelength of 510 nm.

Unless they are defined in another way, all the technical and scientific terms used here have the same meaning as that usually understood by a ordinary specialist of the field to which this invention belongs. Similarly, all the publications, patent applications, all the patents and all other references mentioned here are incorporated by way of reference.

### EXPERIMENTAL PART

A/MBP-Cdc25C Fusion Protein:

1—Construction of the Expression Vector for MBP-Cdc25C.

1.1—Principle of the System Used.

The system used (New England Biolabs #800-pMAL™ fusion protein and purification system) is based on the production of a fusion protein between the protein of interest, here the human Cdc25C protein, and the bacterial protein MBP (Maltose-Binding Protein) of Escherichia coli. This method allows a one stage purification of the fusion protein due to the affinity of the MBP for maltose.

1.2—Origin of the cDNA of Cdc25C

The DNA coding for human Cdc25C phosphatase corresponds to accession number 4502706.

Starting from this plasmid, the DNA coding for Cdc25C was amplified by polymerase chain reaction (PCR) using the C-XBAI SENSE and C-XBAI ANTI-SENSE primers (with sequences SEQ. ID. No.s 3 and 4 respectively) and introduced into the vector pCDNA3-HA at the XbaI site in order to produce the vector Cdc25C pcDNA3-HA. The Cdc25C/XbaI insert was, at this stage, sequenced in its entirety.

The C-XBAI SENSE primer has as its sequence, sequence SEQ. ID. No. 3 represented below:

5′-GTTCTAGAAT GTCTAGAA CTCTTC-3′

The C-XBAI ANTI-SENSE primer has as its sequence, sequence SEQ. ID. No. 4 represented below:

5′-GGCTCTGA GTTGCGC CGG-3′

1.3—Construction of the Vector pMAL—Hs Cdc25C

The vector Cdc25C pcDNA3-HA was digested by XbaI, the Cdc25C/XbaI insert (1456 base pairs) was purified and introduced into the vector pMAL™-c2X (New England Biolabs, #800-76) at the XbaI site, in order to produce the vector pMAL-Hs Cdc25C (with sequence SEQ. ID No. 2). This vector allows the production of a fusion protein MBP-Cdc25C of 868 amino acids, from the Ptac bacterial promotor inducible by isopropylthiogalactoside (IPTG).

The sequence SEQ. ID. No. 2 of the Hs Cdc25C protein with its XBAI restriction ends is the following:

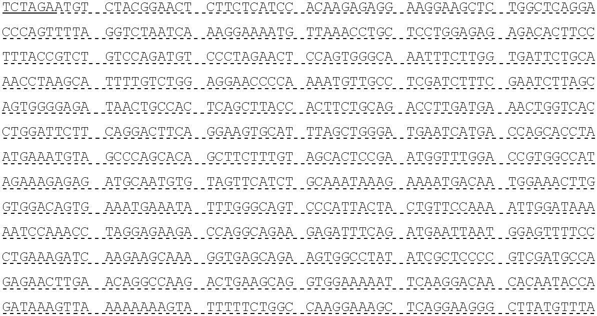

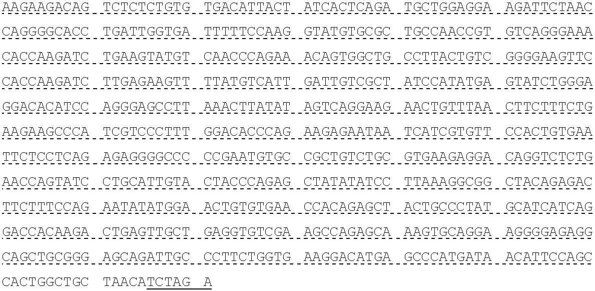

(the XBAI sites are continuously underlined, the open reading frame (or ORF) of Cdc25C is underlined by a dotted line)

1.4—Creation of the JM109/pMAL—Hs Cdc25C Strain

The vector Cdc25C pMAL—Hs was introduced into the Escherichia coli (E. coli) JM109 (Stratagene #200271) strain. An isolated colony was selected and the production of a protein with a theoretical apparent molecular weight of 97 kDa after culture in the presence of IPTG was observed by analysis of the total bacterial proteins on denaturing polyacrylamide gel and staining with Coomassie blue. The identity of the fusion protein was then confirmed by western blot and immunodetection with an anti-Cdc25C antibody.

Finally, the plasmidic DNA isolated from this clone was sequenced in the region corresponding to Cdc25C to verify the absence of mutations or modifications of the sequence which could have been generated during the processes of sub-cloning and/or transformation of DNA.

The following sequence (designated by SEQ. ID. No. 5) was obtained for the JM109/pMAL—Hs Cdc25C strain:

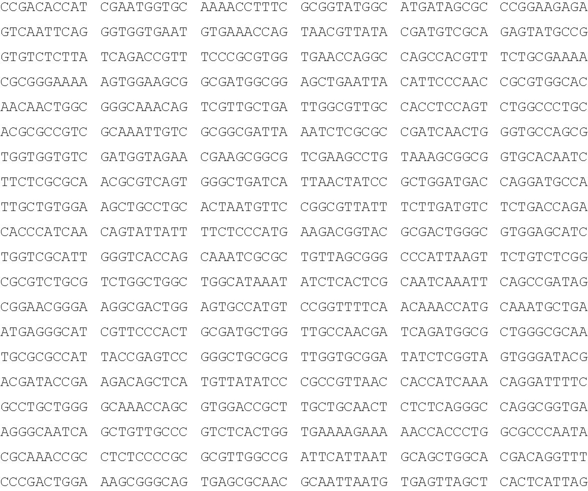

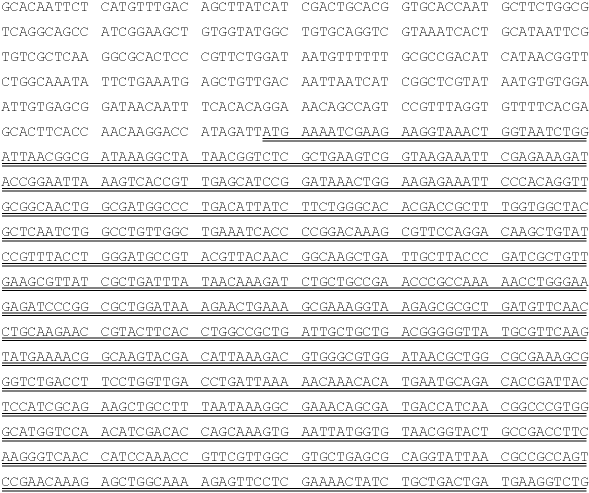

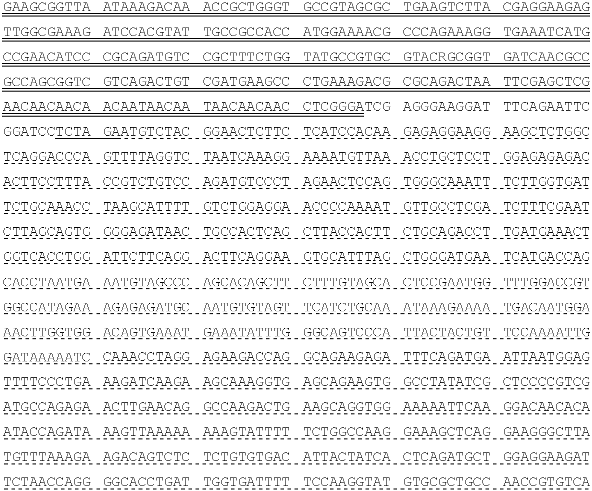

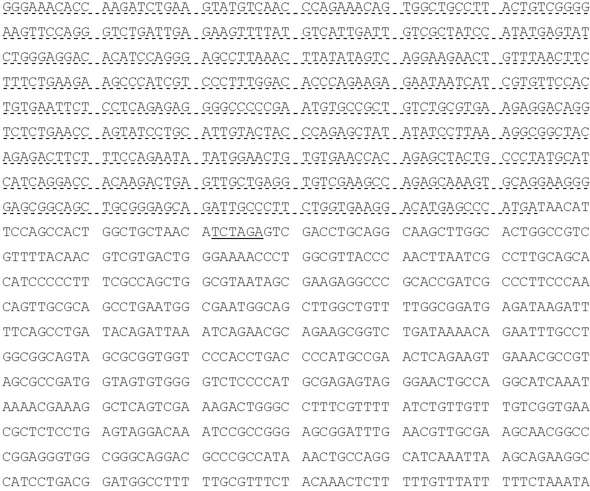

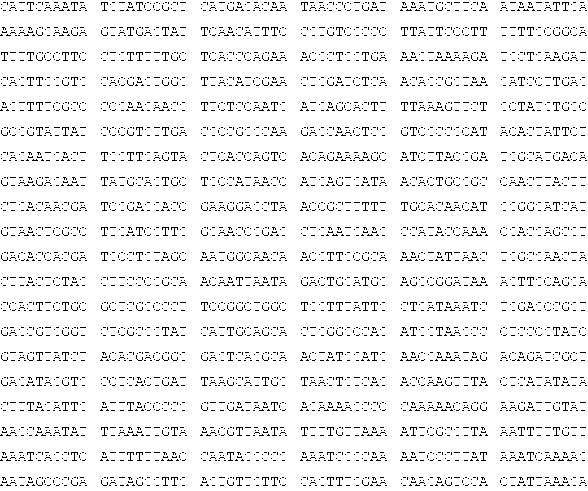

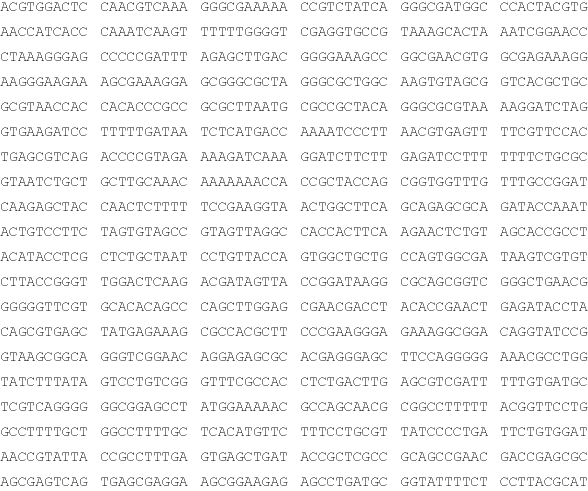

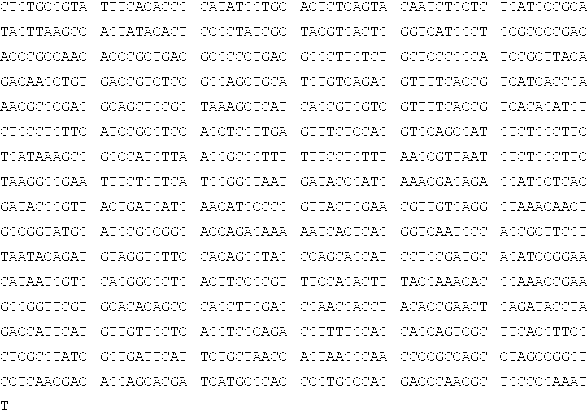

(the XBAI sites are underlined by a single continuous line, the ORF of Cdc25C is underlined by a dotted line, the maltose binding protein (MBP) sequence is underlined by a double continuous line and the sequence in bold corresponds to the Plasmidic DNA region isolated from the JM 109 strain and then sequenced).

This clone can be stored at −80° C. in the form of a saturated culture with added glycerol (final concentration 25%) or “stock glycerol”. This strain will be used for all the subsequent production stages.

2—Production and Purification of the Recombinant MBP-Cdc25C Protein:

NB: except where otherwise indicated, all the chemical reagents are supplied by SIGMA-ALDRICH.

2.1—Bacterial Culture and Induction of the Expression of the Fusion Protein

50 ml of LB medium+ampicillin 100 μg/ml (LB amp.) are inoculated with 100 μl of stock glycerol from the JM109/pMAL-Cdc25C clone and cultured for 14 to 16 hours at 37° C. under stirring (180 to 220 rpm). This pre-culture is then diluted fifty times (20 ml per litre of medium) in an LB amp. medium +2 g/l of glucose and cultured at 37° C./180 rpm in order to reach an optical density at 600 nm of between 0.55 and 0.60. Synthesis of the fusion protein is then induced by adding IPTG (0.3 mM) at 37° C. over 3 hours. The bacteria are collected by centrifugation, washed once in 40 ml of cold PBS per litre of culture, and the bacterial pellet is then frozen in liquid nitrogen and stored at −80° C.

The induction is analysed immediately by depositing 2.5.10⁷ cells, removed before and after induction, on denaturing polyacrylamide gel and staining of the proteins with Coomassie blue (FIG. 1, lines 1 and 2 respectively).

2.2—Lysis and Extraction.

A bacterial pellet corresponding to 1 litre of induced culture is thawed in ice, re-suspended in 35 ml of lysis buffer (20 mM Tris-HCl pH 7.4, 250 mM NaCl, 1 mM EDTA, 1 mM DTT, 10 μg/ml lysozyme, 1 μg/ml leupeptin, 2 μg/ml aprotinin, 1 mM PMSF) and incubated for 45 minutes in ice. The bacterial suspension is then sonicated (4 cycles of 1 min in discontinuous mode 50%, alternated with 1 minutes pause), then centrifuged for 35 minutes at 110 000 g. The supernatant or soluble extract is retained for purification of the MBP-Cdc25C protein (FIG. 1, line 3).

2.3—Affinity Purification on Amylose-Agarose Resin.

For a soluble extract corresponding to 1 litre of induced bacterial culture, 2 ml of amylose-agarose resin (New England Biolabs #800-21) is deposited on an HR 5/10 chromatographic column (Pharmacia) and washed with 20 ml (10 volumes) of column buffer (20 mM Tris-HCl pH 7.4; 250 mM NaCl; 1 mM EDTA; 1 mM DTT; 1 μg/ml leupeptin; 2 μg/ml aprotinin). The soluble extract is passed through the affinity column at a flow rate of 0.15 ml/min; the eluate (i.e. the fraction not retained on the amylose-agarose) is collected for analysis (FIG. 1, line 4). The column is washed with 20 ml (10 volumes) of column buffer. A sample of the affinity matrix after passage of the soluble extract can be optionally analysed (FIG. 1, line 5).

Elution of the protein of the affinity matrix is carried out with a maltose buffer (20 mM Tris-HCl pH 7.4; 250 mM NaCl; 1 mM EDTA; 1 mM DTT; 10 mM maltose). 20 elution fractions of 0.5 ml are collected. For each fraction, the total protein concentration is evaluated by a Bradford type test and the fraction is analysed by depositing on denaturing polyacrylamide gel and staining with Coomassie blue (FIG. 1, line 6). The fractions in which the complete MBP-Cdc25C fusion protein represents at least 90% of the total proteins are collected together to form a batch, the activity of which is then tested. The batches are stored at −80° C.

Sequencing of the insert comprised between the XbaI sites produces the following result:

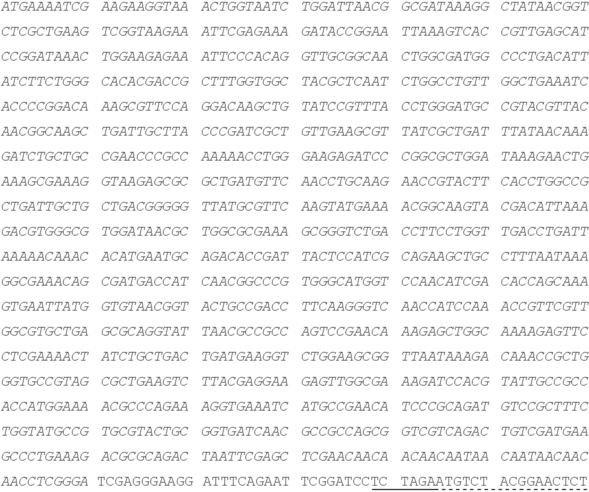

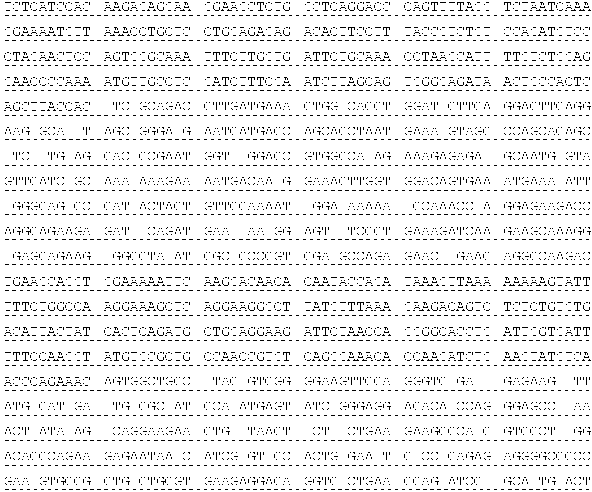

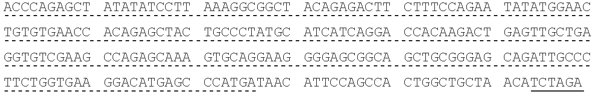

(the XBAI sites are underlined by a single continuous line, the ORF of Cdc25C is underlined by a dotted line and the sequence in italics correspond to the ORF of the maltose binding protein (MBP))

The sequence of the obtained MBP-Cdc25C fusion protein therefore corresponds to the sequence SEQ. ID No. 1 represented below:

ATGAAAATCG AAGAAGGTAA ACTGGTAATC TGGATTAACG
GCGATAAAGG CTATAACGGT CTCGCTGAAG TCGGTAAGAA
ATTCGAGAAA GATACCGGAA TTAAAGTCAC CGTTGAGCAT
CCGGATAAAC TGGAAGAGAA ATTCCCACAG GTTGCGGCAA
CTGGCGATGG CCCTGACATT ATCTTCTGGG CACACGACCG
CTTTGGTGGC TACGCTCAAT CTGGCCTGTT GGCTGAAATC
ACCCCGGACA AAGCGTTCCA GGACAAGCTG TATCCGTTTA
CCTCGGATGC CGTACGTTAC AACGGCAAGC TGATTGCTTA
CCCGATCGCT GTTGAAGCGT TATCGCTGAT TTATAACAAA
GATCTGCTGC CGAACCCGCC AAAAACCTGG GAAGAGATCC
CGGCGCTGGA TAAAGAACTG AAAGCGAAAG GTAAGAGCGC
GCTGATGTTC AACCTGCAAG AACCGTACTT CACCTGGCCG
CTGATTGCTG CTGACCGGGG TTATGCGTTC AAGTATGAAA
ACGGCAAGTA CGACATTAAA GACGTGGGCG TGGATAACGC
TGGCGCGAAA GCGGGTCTGA CCTTCCTGGT TGACCTGATT
AAAAACAAAC ACATGAATGC AGACACCGAT TACTCCATCG
CAGAAGCTGC CTTTAATAAA GGCGAAACAG CGATGACCAT
CAACGGCCCG TGGGCATGGT CCAACATCGA CACCAGCAAA
GTGAATTATG GTGTAACGGT ACTGCCGACC TTCAAGGGTC
AACCATCCAA ACCGTTCGTT GGCGTGCTGA GCGCAGGTAT
TAACGCCGCC AGTCCGAACA AAGAGCTGGC AAAAGAGTTC
CTCGAAAACT ATCTGCTGAC TGATGAAGGT CTGGAAGCGG
TTAATAAAGA CAAACCGCTG GGTGCCGTAG CGCTGAAGTC
TTACGAGGAA GAGTTGGCGA AAGATCCACG TATTGCCGCC
ACCATGGAAA ACGCCCAGAA AGGTGAAATC ATGCCGAACA
TCCCGCAGAT GTCCGCTTTC TGGTATGCCG TGCGTACTGC
GGTGATCAAC GCCGCCAGCG GTCGTCAGAC TGTCGATGAA
GCCCTGAAAG ACGCGCAGAC TAATTCGAGC TCGAACAACA
ACAACAATAA CAATAACAAC AACCTCGGGA TCGAGGGAAGG
ATTTCAGAAT TCGGATCCTC TAGAATGTCT ACGGAACTCT
TCTCATCCAC AAGAGAGGAA GGAAGCTCTG GCTCAGGACC
CAGTTTTAGG TCTAATCAAA GGAAAATGTT AAACCTGCTC
CTGGAGAGAG ACACTTCCTT TACCGTCTGT CCAGATGTCC
CTAGAACTCC AGTGGGCAAA TTTCTTGGTG ATTCTGCAAA
CCTAAGCATT TTGTCTGGAG GAACCCCAAA ATGTTGCCTC
GATCTTTCGA ATCTTAGCAG TGGGGAGATA ACTGCCACTC
AGCTTACCAC TTCTGCAGAC CTTGATGAAA CTGGTCACCT
GGATTCTTCA GGACTTCAGG AAGTGCATTT AGCTGGGATG
AATCATGACC AGCACCTAAT GAAATGTAGC CCAGCACAGC
TTCTTTGTAG CACTCCGAAT GGTTTGGACC GTGGCCATAG
AAAGAGAGAT GCAATGTGTA GTTCATCTGC AAATAAAGAA
AATGACAATG GAAACTTGGT GGACAGTGAA ATGAAATATT
TGGGCAGTCC CATTACTACT GTTCCAAAAT TGGATAAAAA
TCCAAACCTA CCAGAAGACC AGGCAGAAGA GATTTCAGAT
GAATTAATGG AGTTTTCCCT GAAAGATCAA CAAGCAAAGG
TGAGCAGAAG TGGCCTATAT CGCTCCCCGT CGATGCCAGA
GAACTTGAAC AGGCCAAGAC TGAAGCAGGT GGAAAAATTC
AAGGACAACA CAATACCACA TAAAGTTAAA AAAAAGTATT
TTTCTGGCCA ACGAAAGCTC AGGAAGGCCT TATGTTTAAA
GAAGACAGTC TCTCTGTGTG ACATTACTAT CACTCAGATG
CTGGAGGAAG ATTCTAACCA GGGGCACCTG ATTGGTGATT
TTTCCAAGGT ATGTGCGCTG CCAACCGTGT CAGGGAAACA
CCAAGATCTG AAGTATGTCA ACCCAGAAAC AGTGGCTGCC
TTACTGTCGG GGAAGTTCCA GGGTCTGATT GAGAAGTTTT
ATGTCATTGA TTGTCGCTAT CCATATGAGT ATCTGGGAGG
ACACATCCAG GGAGCCTTAA ACTTATATAG TCAGGAAGAA
CTGTTTAACT TCTTTCTGAA GAAGCCCATC GTCCCTTTGG
ACACCCAGAA GAGAATAATC ATCGTGTTCC ACTGTGAATT
CTCCTCAGAG AGGGGCCCCC GAATGTGCCG CTGTCTGCGT
GAAGAGGACA GGTCTCTGAA CCAGTATCCT GCATTGTACT
ACCCAGAGCT ATATATCCTT AAAGGCGGCT ACAGAGACTT
CTTTCCAGAA TATATGGAAC TGTGTGAACC ACAGAGCTAC
TGCCCTATGC ATCATCAGGA CCACAAGACT GAGTTGCTGA
GGTGTCGAAG CCAGAGCAAA GTGCAGGAAG GGGAGCGGCA
GCTGCGGGAG CAGATTGCCC TTCTGGTGAA GGACATGAGC
CCATGA

3—Determination of the Activity of the MBP-Cdc25C Fusion Protein:

The phosphatase activity of the MBP-Cdc25C protein is evaluated by a test of the dephosphorylation of 3-O-methylfluoresceine phosphate (OMFP) with determination of the absorbance at 477 nm (OD 477 nm) of the product of the reaction (OMF).

The MBP-Cdc25C protein, stored in elution buffer (the same as that described in paragraph 2.3), is diluted to the concentration of 20 nM in the phosphatase reaction buffer (50 mM Tris-HCl pH 8.2; 50 mM NaCl; 1 mM DTT; 20% glycerol), at ambient temperature, in a total reaction volume of 1 ml. The reaction is initiated by the addition of a 0.3 mM solution of OMFP (prepared extemporaneously from a 7.5 mM stock solution in 100% DMSO (Sigma #M2629)) and takes place at 25° C. in a disposable spectrophotometry polystyrene cuvet (Fisher Scientific #A12-103-056). The OD 477 nm is measured after 90 minutes. The reference for the absorbance measurement is constituted by the reaction buffer containing 0.3 mM of OMFP, without MBP-Cdc25C protein, at time t₀ of the reaction. A representative example of the results of such a determination of activity is illustrated by FIG. 2.

B/MBP-Cdc25B1, MBP-Cdc25B2 and MBP-Cdc25B3 Fusion Proteins:

1—Construction of the Expression Vector for MBP-Cdc25B1, MBP-Cdc25B2 and MBP-Cdc25B3

1.1—Principle of the System Used.

The system used (New England Biolabs #800-pMAL™ fusion protein and purification system) is based on the production of a fusion protein between the protein of interest, here the human protein Cdc25B, and the bacterial protein MBP (Maltose-Binding Protein) of Escherichia coli. This method allows a one stage purification of the fusion protein due to the affinity of the MBP for maltose.

1.2—Origin of the cDNA of Cdc25B

The DNA coding for the three splicing variants of human Cdc25B phophatase corresponds to the accession numbers: M81934 and Z68092.

1.3—Construction of the Vectors pMAL-Hs Cdc25B

The differences between the DNA of the 3 Cdc25B variants concern two exons coding for domains of 14 and 42 amino acids located in the regulatory region of the molecule (Baldin et al., Oncogene (1997), 14, 2485-2490) and does not affect its 5′ and 3′ regions. The cloning strategy used for these three variants is therefore identical.

The cDNA of Cdc25B in the form of NdeI(Klenow)/BamHI inserts were introduced into the pMAL™-C2X vector (New England Biolabs, #800-76) at the EcoR1 (Klenow)/BamH1 site in order to produce the pMAL-Hs Cdc25B1, pMAL-Hs Cdc25B2 and pMAL-Hs Cdc25B3 vectors respectively. These vectors allow the production of the MBP-Cdc25B1 (962 amino acids), MPB-Cdc25B2 (933 amino acids) and MBP-Cdc25B3 (974 amino acids) fusion proteins from the Ptac bacterial promotor inducible by isopropylthiogalactoside (IPTG).

The sequence SEQ. ID. No. 6 of the Hs Cdc25B1 protein with its NdeI and BamH1 restriction ends is the following:

CATATGGAGG TGCCCCAGCC GGAGCCCGCG CCAGGCTCGG
CTCTCAGTCC AGCAGCCGTG TGCGGTGGCG CCCAGCGTCC
GGGCCACCTC CCGGGCCTCC TGCTGGGATC TCATGGCCTC
CTGGGGTCCC CGGTGCGGGC GGCCGCTTCC TCGCCGGTCA
CCACCCTCAC CCAGACCATG CACGACCTCG CCGGGCTCGG
CAGCCGCAGC CGCCTGACGC ACCTATCCCT GTCTCGACGG
GCATCCGAAT CCTCCCTGTC GTCTGAATCC TCCGAATCTT
CTGATGCAGG TCTCTGCATG GATTCCCCCA GCCCTATGGA
CCCCCACATG GCGGAGCAGA CGTTTGAACA GGCCATCCAG
GCAGCCAGCC GGATCATTCG AAACGAGCAG TTTGCCATCA
GACGCTTCCA GTCTATGCCG GTGAGGCTGC TGGGCCACAG
CCCCGTGCTT CGGAACATCA CCAACTCCCA GGCGCCCGAC
GGCCGGAGGA AGAGCGAGGC GGGCAGTGGA GCTGCCAGCA
GCTCTGGGGA AGACAAGGAG AATGATGGAT TTGTCTTCAA
GATGCCATGG AAGCCCACAC ATCCCAGCTC CACCCATGCT
CTGGCAGAGT GGGCCAGCCG CAGGGAAGCC TTTGCCCAGA
GACCCAGCTC GGCCCCCGAC CTGATGTGTC TCAGTCCTGA
CCGGAAGATG GAAGTGGAGG AGCTCAGCCC CCTGGCCCTA
GGTCGCTTCT CTCTGACCCC TGCAGAGGGG GATACTCAGG
AAGATGATGG ATTTGTGGAC ATCCTAGAGA GTGACTTAAA
GGATGATGAT GCAGTTCCCC CAGGCATGGA GAGTCTCATT
AGTGCCCCAC TGGTCAAGAC CTTGGAAAAG GAAGAGGAAA
AGGACCTCGT CATGTACAGC AAGTGCCAGC GGCTCTTCCG
CTCTCCGTCC ATGCCCTGCA GCGTGATCCG GCCCATCCTC
AAGAGGCTGG AGCGGCCCCA GGACAGGGAC ACGCCCGTGC
AGAATAAGCG GAGGCGGAGC GTGACCCCTC CTGAGCAGCA
GCAQGAGGCT GAGGAACCTA AAGCCCGCGT CCTCCGCTCA
AAATCACTGT GTCACGATGA GATCGAGAAC CTCCTGGACA
GTGACCACCG AGAGCTGATT GGAGATTACT CTAAGGCCTT
CCTCCTACAG ACAGTAGACG GAAAGCACCA AGACCTCAAG
TACATCTCAC CAGAAACGAT GGTGGCCCTA TTGACGGGCA
AGTTCAGCAA CATCGTGGAT AAGTTTGTGA TTGTAGACTG
CAGATACCCC TATGAATATG AAGGCGGGCA CATCAAGACT
GCGGTGAACT TGCCCCTGGA ACGCGACGCC GAGAGCTTCC
TACTGAAGAG CCCCATCGCG CCCTGTAGCC TGGACAAGAG
AGTCATCCTC ATTTTCCACT GTGAATTCTC ATCTGAGCGT
GGGCCCCGCA TGTGCCGTTT CATCAGGGAA CGAGACCGTG
CTGTCAACGA CTACCCCAGC CTCTACTACC CTGAGATGTA
TATCCTGAAA GGCGGCTACA AGGAGTTCTT CCCTCAGCAC
CCGAACTTCT GTGAACCCCA GGACTACCGG CCCATGAACC
ACGAGGCCTT CAAGGATGAG CTAAAGACCT TCCGCCTCAA
GACTCGCAGC TGGGCTGGGG AGCGGAGCCG GCGGGAGCTC
TGTAGCCGGC TGCAGGACCA GTGAGGGGCC TGCGCCAGTC
CTGCTACCTC CCTTGCCTTT CGAGGCCTGA AGCCAGCTGC
CCTATGGGCC TCCCGGGCTG AGGGCCTCCT GGAGGCCTCA
GGTGCTGTCC ATGGGAAAGA TGGTGTGGTG TCCTGCCTGT
CTGCCCCAGC CCAGATTCCC CTGTGTCATC CCATCATTTT
CCATATCCTG GTGCCCCCCA CCCCTGGAAG AGCCCAGTCT
GTTGAGTTAG TTAAGTTGGG TTAATACCAG CTTAAAGGCA
GTATTTTGTG TCCTCCAGGA GCTTCTTGTT TCCTTGTTAG
GGTTAACCCT TCATCTTCCT GTGTCCTGAA ACGCTCCTTT
GTGTGTGTGT CAGCTGAGGA TCC

(the NdeI and BamH1 sites are continuously underlined)

The sequence SEQ. ID. No. 7 of the Hs Cdc25B2 protein with its NdeI and BamH1 restriction ends is the following:

CATATGGAGG TGCCCCAGCC GGAGCCCGCG CCAGGCTCGG
CTCTCAGTCC AGCAGGCGTG TGCGGTGGCG CCCAGCGTCC
GGGCCACCTC CCGGGCCTCC TGCTGGGATC TCATGGCCTC
CTGGGGTCCC CGGTGCGGGC GGCCGCTTCC TCGCCGGTCA
CCACCCTCAC CCAGACCATG CACGACCTCG CCGGGCTCGG
CAGCGAAACC CCAAAGAGTC AGOTAGGGAC CCTGCTCTTC
CGCAGCCGCA GCCGCCTGAC GCACCTATCC CTGTCTCGAC
GGGCATCCGA ATCCTCCCTG TCGTCTGAAT CCTCCGAATC
TTCTGATGCA GGTCTCTGCA TGGATTCCCC CAGCCCTATG
GACCCCCACA TGGCGGAGCA GACGTTTGAA CAGGCCATCC
AGGCAGCCAG CCGGATCATT CGAAACGAGC AGTTTGCCAT
CAGACGCTTC CAGTCTATGC CGGATGGATT TGTCTTCAAG
ATGCCATGGA AGCCCACACA TCCCAGCTCC ACCCATGCTC
TGGCAGAGTG GGCCAGCCGC AGGGAAGCCT TTGCCCAGAG
ACCCAGCTCG GCCCCCGACC TGATGTGTCT CAGTCCTGAC
CGGAAGATGG AAGTGGAGGA GCTCAGCCCC CTGGCCCTAG
GTCGCTTCTC TCTGACCCCT GCAGAGGGGG ATACTGAGGA
AGATGATGGA TTTGTGGACA TCCTAGAGAG TGACTTAAAG
GATGATGATG CAGTTCCCCC AGGCATGGAG AGTCTCATTA
GTGCCCCACT GGTCAAGACC TTGGAAAAGG AAGAGGAAAA
GGACCTCGTC ATGTACAGCA AGTGCCAGCG GCTCTTCCGC
TCTCCGTCCA TGCCCTGCAG CGTGATCCGG CCCATCCTCA
AGAGGCTGGA GCGGCCCCAG GACAGGGACA CGCCCGTGCA
GAATAAGCGG AGGCGGAGCG TGACCCCTCC TGAGGAGCAG
CAGGAGGCTG AGGAACCTAA AGCCCGCGTC CTCCGCTCAA
AATCACTGTG TCACGATGAG ATCGAGAACC TCCTGGACAG
TGACCACCGA GAGCTGATTG GAGATTACTC TAAGGCCTTC
CTCCTACAGA CAGTAGACGG AAAGCACCAA GACCTCAAGT
ACATCTCACC AGAAACGATG GTGGCCCTAT TGACGGGCAA
GTTCAGCAAC ATCGTGGATA AGTTTGTGAT TGTAGACTGC
AGATACCCCT ATGAATATGA AGGCGGGCAC ATCAAGACTG
CGGTGAACTT GCCCCTGGAA CGCGACGCCG AGAGCTTCCT
ACTGAAGAGC CCCATCGCGC CCTGTAGCCT GGACAAGAGA
GTCATCCTCA TTTTCCACTG TGAATTCTCA TCTGAGCGTG
GGCCCCGCAT GTGCCGTTTC ATCAGGGAAC GAGACCGTGC
TGTCAACGAC TACCCCAGCC TCTACTACCC TGAGATGTAT
ATCCTGAAAG GCGGCTACAA GGAGTTCTTC CCTCAGCACC
CGAACTTCTG TGAACCCCAG GACTACCGGC CCATGAACCA
CGAGGCCTTC AAGGATGAGC TAAAGACCTT CCGCCTCAAG
ACTCGCAGCT GGGCTGGGGA GCGGAGCCGG CGGGAGCTCT
GTAGCCGGCT GCAGGACCAG TGAGGGGCCT GCGCCAGTCC
TGCTACCTCC CTTGCCTTTC GAGGCCTGAA GCCAGCTGCC
CTATGGGCCT GCCGGGCTGA GGGCCTGCTG GAGGCCTCAG
GTGCTGTCCA TGGGAAAGAT GGTGTGGTGT CCTGCCTGTC
TGCCCCAGCC CAGATTCCCC TGTGTCATCC CATCATTTTC
CATATCCTGG TGCCCCCCAC CCCTGGAAGA GCCCAGTCTG
TTGAGTTAGT TAAGTTGGGT TAATACCAGC TTAAAGGCAG
TATTTTGTGT CCTCCAGGAG CTTCTTGTTT CCTTGTTAGG
GTTAACCCTT CATCTTCCTG TGTCCTGAAA CGCTCCTTTG
TGTGTGTGTC AGCTGAGGAT CC

(the NdeI and BamH1 sites are continuously underlined)

The sequence SEQ. ID. No. 8 of the Hs Cdc25B3 protein with its NdeI and BamH1 restriction ends is the following:

CATATGGAGG TGCCCCAGCC GGAGCCCGCG CCAGGCTCGG
CTCTCAGTCC AGCAGGCGTG TGCGGTGGCG CCCAGCGTCC
GGGCCACCTC CCGGGCCTCC TGCTGGGATC TCATGGCCTC
CTGGGGTCCC CGGTGCGGGC GGCCGCTTCC TCGCCGGTCA
CCACCCTCAC CCAGACCATG CACGACCTCG CCGGGCTCGG
CAGCGAAACC CCAAAGAGTC AGGTAGCGAC CCTGCTCTTC
CGCAGCCGCA GCCGCCTGAC GCACCTATCC CTGTCTCGAC
GGGCATCCGA ATCCTCCCTG TCGTCTGAAT CCTCCGAATC
TTCTGATGCA GGTCTCTGCA TGGATTCCCC CAGCCCTATG
GACCCCCACA TGGCGGAGCA GACGTTTGAA CAGGCCATCC
AGGCAGCCAG CCGGATCATT CGAAACGACC AGTTTGCCAT
CAGACGCTTC CAGTCTATGC CGGTGAGGCT GCTGGGCCAC
AGCCCCGTCC TTCGGAACAT CACCAACTCC CAGGCGCCCG
ACGGCCGGAG GAAGAGCGAG GCGGGCAGTG GAGCTGCCAG
CAGCTCTGGG GAAGACAAGG AGAATGATGG ATTTGTCTTC
AAGATGCCAT GGAAGCCCAC ACATCCCAGC TCCACCCATG
CTCTGGCAGA GTGGGCCAGC CGCAGGGAAG CCTTTGCCCA
GAGACCCAGC TCGGCCCCCG ACCTGATGTG TCTCAGTCCT
GACCGGAAGA TGGAAGTGGA GGAGCTCAGC CCCCTGGCCC
TAGGTCGCTT CTCTCTGACC CCTGCAGAGG GGGATACTGA
GGAAGATGAT GGATTTGTGG ACATCCTAGA GAGTGACTTA
AAGGATGATG ATGCAGTTCC CCCAGGCATG GAGAGTCTCA
TTAGTGCCCC ACTGGTCAAG ACCTTGGAAA AGGAAGAGGA
AAAGGACCTC GTCATGTACA GCAAGTGCCA GCGGCTCTTC
CGCTCTCCGT CCATGCCCTG CAGCGTGATC CGGCCCATCC
TCAAGAGGCT GGAGCGGCCC CAGGACAGGG ACACGCCCGT
GCAGAATAAG CGGAGGCGGA GCGTGACCCC TCCTGAGGAG
CAGCAGGAGG CTGAGGAACC TAAAGCCCGC GTCCTCCGCT
CAAAATCACT GTGTCACGAT GAGATCGAGA ACCTCCTGGA
CAGTGACCAC CGAGAGCTGA TTGGAGATTA CTCTAAGCCC
TTCCTCCTAC AGACAGTAGA CGGAAAGCAC CAAGACCTCA
AGTACATCTC ACCAGAAACG ATGGTGGCCC TATTGACGCG
CAAGTTCAGC AACATCGTGG ATAAGTTTGT GATTGTAGAC
TGCAGATACC CCTATGAATA TGAAGGCGGG CACATCAAGA
CTGCGGTGAA CTTGCCCCTG GAACGCGACG CCGAGAGCTT
CCTACTGAAG AGCCCCATCG CGCCCTGTAG CCTGGACAAG
AGAGTCATCC TCATTTTCCA CTGTGAATTC TCATCTGAGC
GTGGGCCCCG CATGTGCCGT TTCATCAGGG AACGAGACCG
TGCTGTCAAC GACTACCCCA GCCTCTACTA CCCTGAGATG
TATATCCTGA AAGGCGGCTA CAAGGAGTTC TTCCCTCAGC
ACCCGAACTT CTGTGAACCC CAGGACTACC GGCCCATGAA
CCACGAGGCC TTCAAGGATG AGCTAAAGAC CTTCCGCCTC
AAGACTCGCA GCTGGGCTGG GGAGCGGAGC CGGCGGGAGC
TCTGTAGCCG GCTGCAGGAC CAGTGAGGGG CCTGCGCCAG
TCCTGCTACC TCCCTTGCCT TTCGAGGCCT GAAGCCAGCT
GCCCTATGGG CCTGCCGGGC TGAGGGCCTG CTGGAGGCCT
CAGGTGCTGT CCATGGGAAA GATGGTGTGC TGTCCTGCCT
GTCTGCCCCA GCCCAGATTC CCCTGTGTCA TCCCATCATT
TTCCATATCC TGGTGCCCCC CACCCCTGGA AGAGCCCAGT
CTGTTGAGTT AGTTAAGTTG GGTTAATACC AGCTTAAAGG
CAGTATTTTG TGTCCTCCAG GAGCTTCTTG TTTCCTTGTT
AGGGTTAACC CTTCATCTTC CTGTGTCCTG AAACGCTCCT
TTGTGTGTGT GTCAGCTGAG GATCC

(the NdeI and BamH1 sites are continuously underlined)

1.4—Creation of the JM109/pMAL-Cdc25B1, B2 and B3 Strains

The vectors pMAL-Cdc25B1-3 were each introduced into the Escherichia coli JM109 strain (Stratagene #200271). The colonies were selected on the basis of their ability to produce a fusion protein after culture in the presence of IPTG. The identity of the proteins was verified by immunodetection with polyclonal antibodies directed against Cdc25B.

The plasmidic DNA isolated from these three clones was sequenced in the region corresponding to Cdc25B. The following sequences were obtained (the part with double underlining corresponds to the ORF of MBP and the part underlined by a dotted line to the ORF of Cdc25B1, Cdc25B2 or Cdc25B3):

SEQ. ID No. 9 (pMAL-HsCdc25B1):

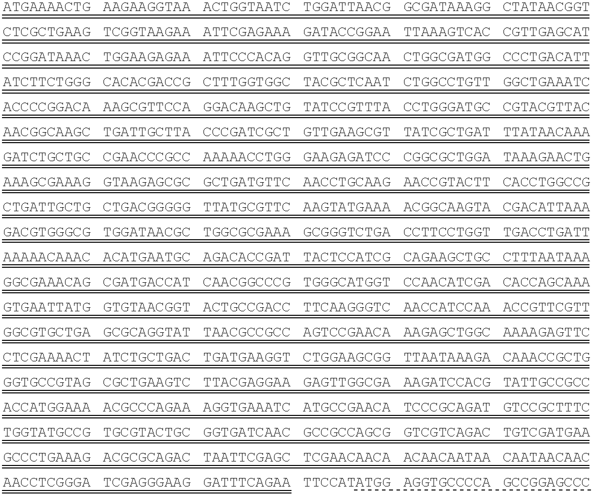

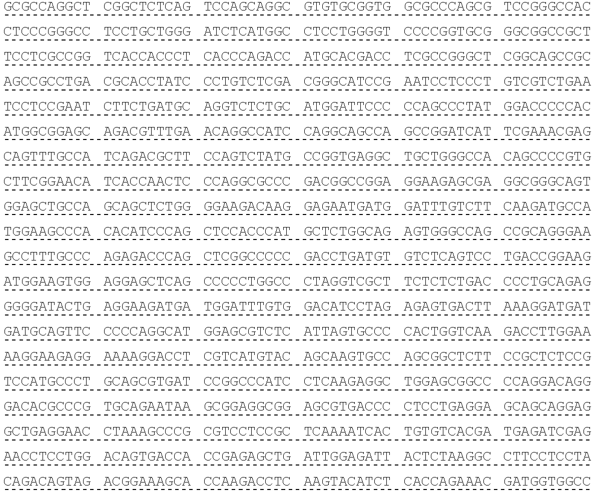

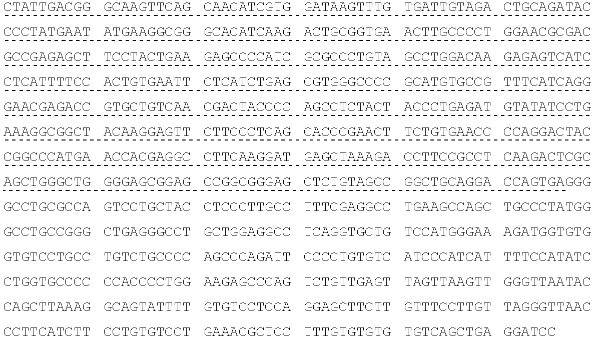

SEQ. ID No. 10 (pMAL-HsCdc25B2):

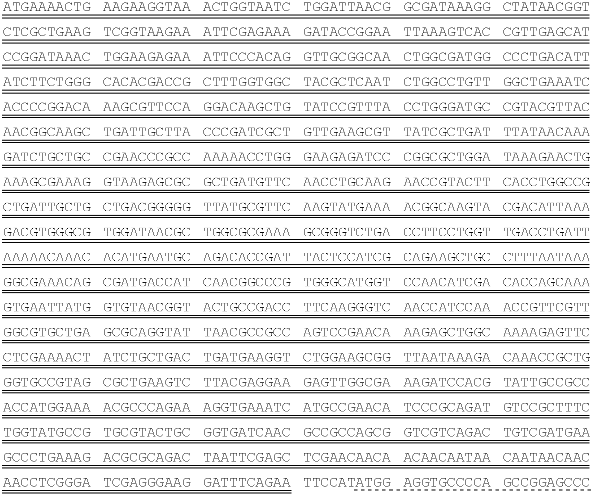

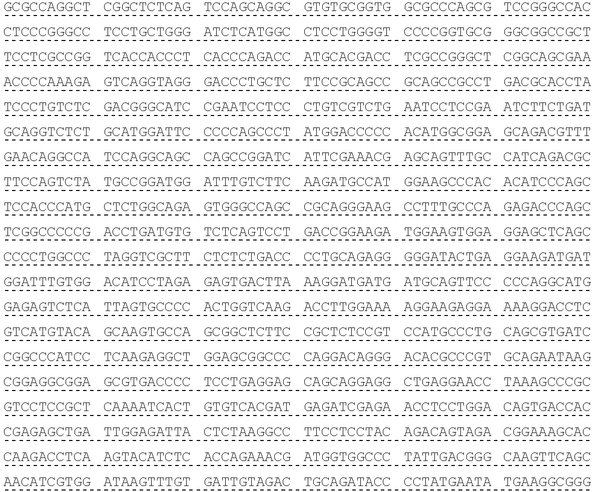

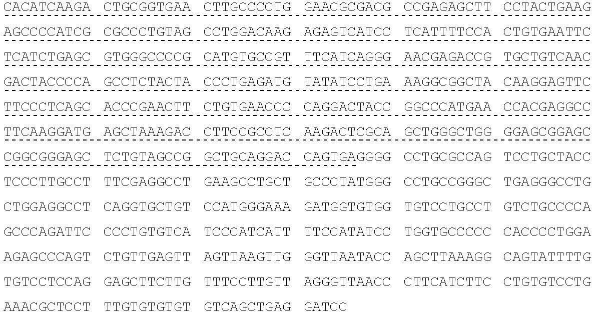

SEQ. ID No. 11 (pMAL-HsCdc25B3):

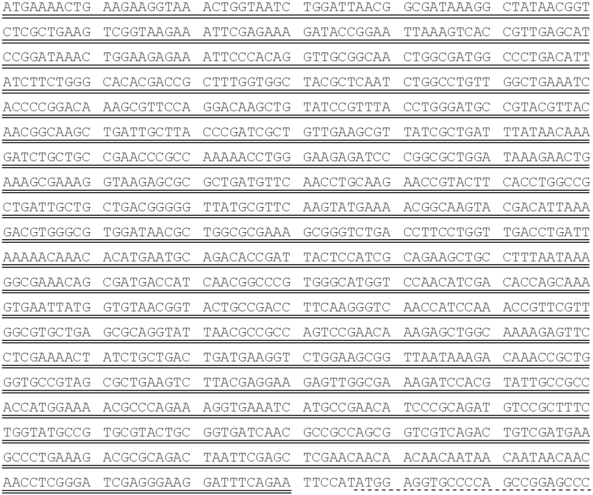

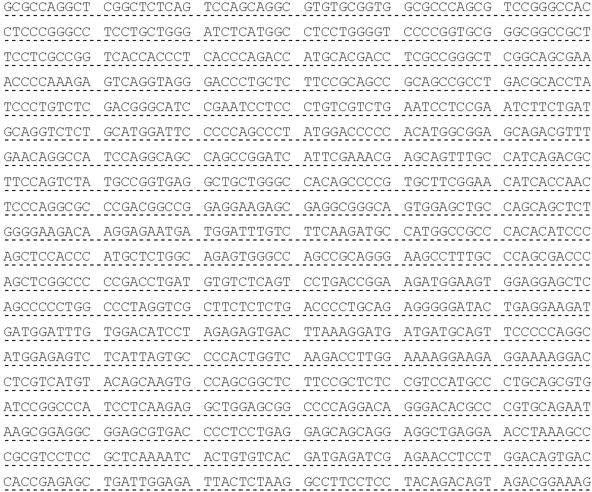

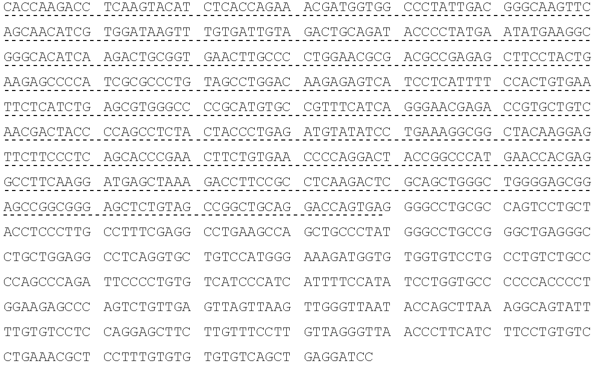

These clones are kept at −80° C. in the form of saturated culture with added glycerol (final concentration 25%). These strains are then used for all the subsequent production stages.

2—Production and Purification of the Recombinant MBP-Cdc25B1, MBP-Cdc25B2 and MBP-Cdc25B3 Proteins:

The recombinant MBP-Cdc25B1, B2 and B3 proteins are produced in exactly the same way as described for MBP-Cdc25C.

The sequences SEQ. ID No. 12, SEQ. ID No. 13 and SEQ. ID No. 14 are obtained for the MBP-Cdc25B1, MBP-Cdc25B2 and MBP-Cdc25B3 fusion proteins respectively:

SEQ. ID No. 12 (MBP-Cdc25B1):

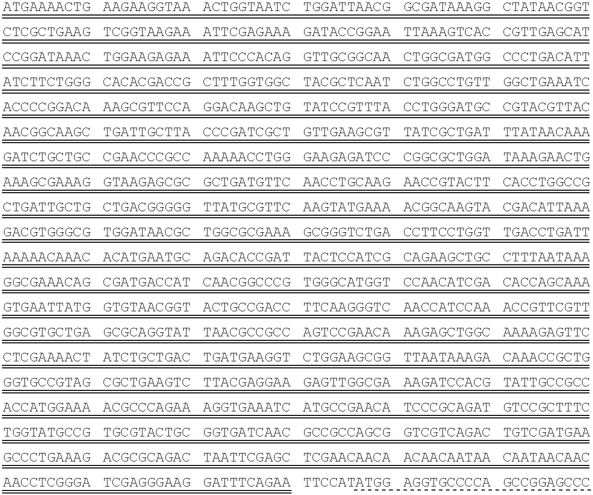

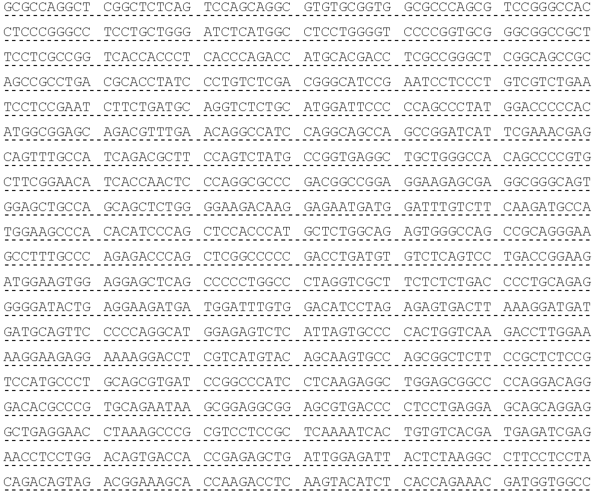

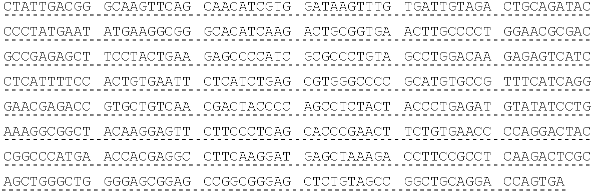

SEQ. ID No. 13 (MBP-Cdc25B2):

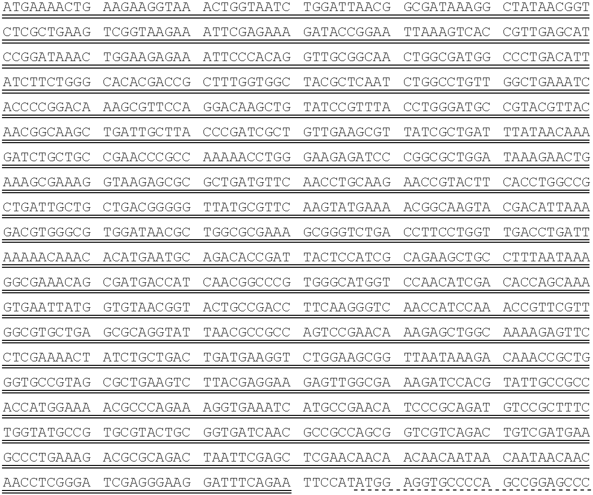

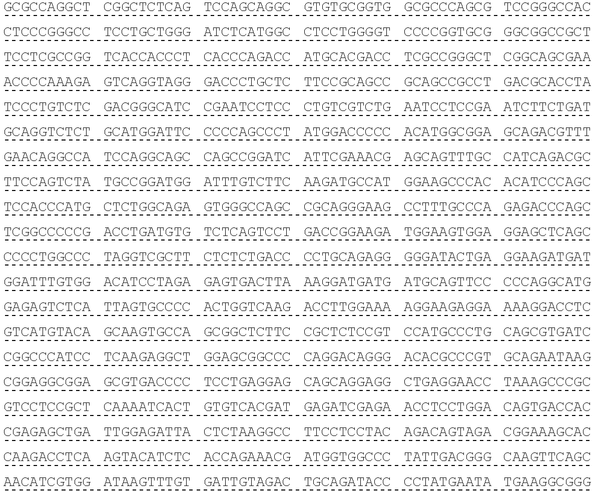

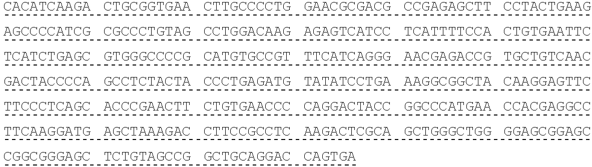

SEQ. ID No. 14 (MBP-Cdc25B3):

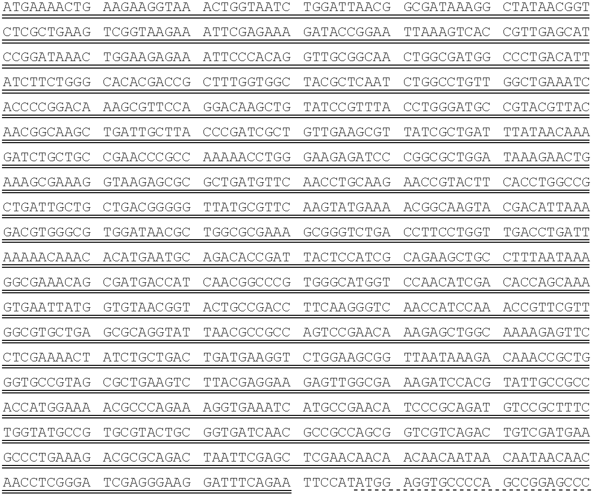

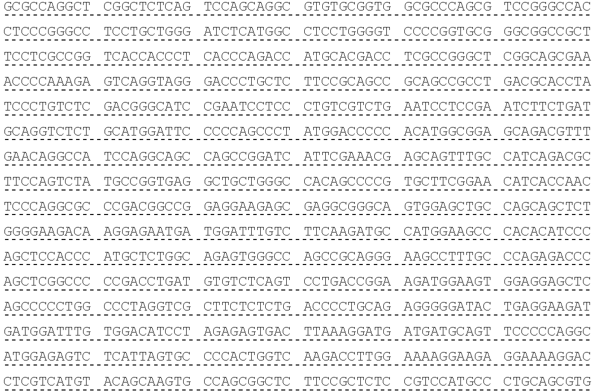

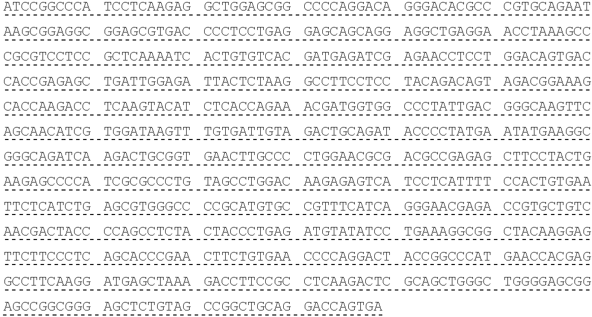

3—Determination of the Activity of the MBP-Cdc25B1, MBP-Cdc25B2 and MBP-Cdc25B3 Fusion Proteins:

The activities of the MBP-Cdc25B1, MBP-Cdc25B2 and MBP-Cdc25B3 fusion proteins are measured under the same conditions as those described for the MBP-Cdc25C fusion protein. The results obtained are given in FIG. 3.

### LEGEND OF THE FIGURES

FIG. 1 (FIG. 1) represents the analysis chromatography relative to the induction of expression of the MBP-Cdc25C fusion protein. Lines 1 and 2 of FIG. 1 correspond respectively to the total JM109/pMAL-Cdc25C extract with or without the addition of IPTG. Line 3 corresponds to the soluble extract. Lines 4 and 5 correspond to the non-retained and retained fractions on amylose-agarose respectively. Finally, line 6 of FIG. 1 corresponds to elution fraction No. 12 which contains practically only fusion protein.

FIG. 2 (FIG. 2) represents the results of measuring the activity of the recombinant MBP-Cdc25C protein (a “+” signifying that menadione had been added to the sample, a “−” signifying that the sample was not treated with menadione).

FIG. 3 (FIG. 3) represents the results of measuring the activity of the recombinant MBP-Cdc25B1, MBP-Cdc25B2 and MBP-Cdc25B3 proteins. The reaction is carried out with 300 ng of enzyme per trial. MBP at the same concentration is used in the control. The fluorescence values measured allow the calculation of the slopes: 0.0025 Δfluo/sec for the control, 0.0361 Δfluo/sec for Cdc25B1, 0.0350 Δfluo/sec for Cdc25B2 and 0.0372 Δfluo/sec for Cdc25B3.

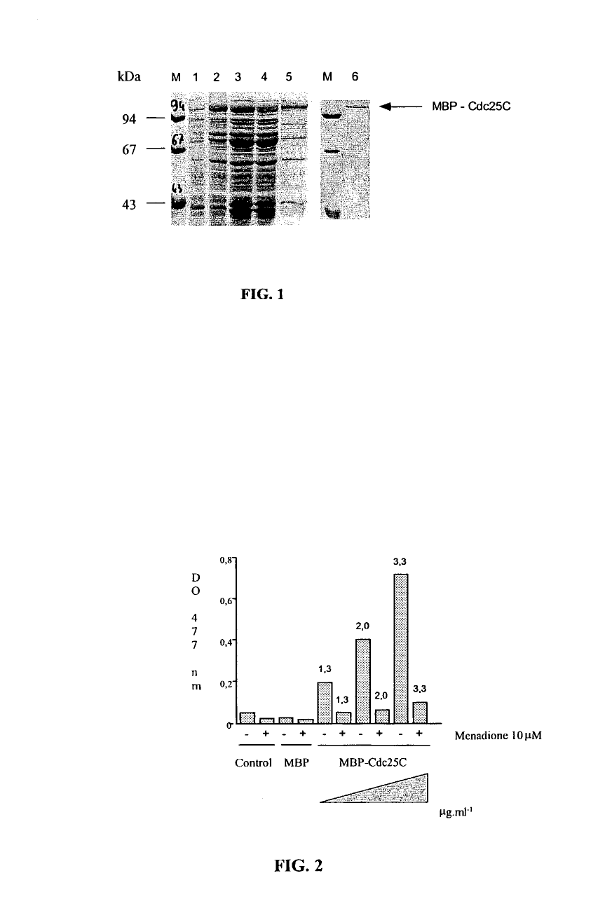

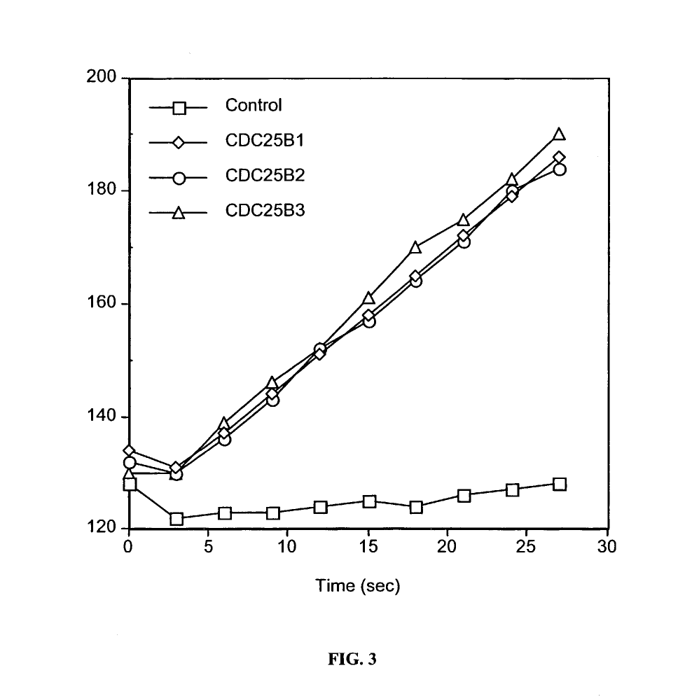

1. Protein characterized in that it is a fusion protein between the maltose binding protein (MBP) and a protein chosen from the Cdc25B1, Cdc25B2, Cdc25B3 and Cdc25C proteins.

2. Protein according to claim 1, characterized in that it is chosen from the following proteins:
a fusion protein between human Cdc25B1 phophatase and the MBP, which is coded by the sequence SEQ. ID No. 12;
a fusion protein between human Cdc25B2 phophatase and the MBP, which is coded by the sequence SEQ. ID No. 13;
a fusion protein between human Cdc25B3 phophatase and the MBP, which is coded by the sequence SEQ. ID No. 14; and
a fusion protein between human Cdc25C phosphatase and the MBP, which is characterized in that it is coded by the sequence SEQ. ID No. 1.

3. Protein according to claim 2, characterized in that it is coded by the sequence SEQ. ID No. 1.

4. DNA characterized in that it codes for a protein according to one of claims 1 to 3.

5. Complementary DNA to the DNA according to claim 4.

6. Bacterial strain characterized in that it is a JM 109 bacterial strain transfected by a plasmid chosen from the plasmid of sequence SEQ. ID No. 9, the plasmid of sequence SEQ. ID No. 10, the plasmid of sequence SEQ. ID No. 11 and the plasmid of sequence SEQ. ID No. 5.

7. Process for the preparation of a protein according to claim 1, characterized in that it comprises the following successive stages:
culture of the bacterial strain JM 109 transfected by the plasmid of sequence SEQ. ID No. 9, the plasmid of sequence SEQ. ID No. 10, the plasmid of sequence SEQ. ID No. 11 or the plasmid of sequence SEQ. ID No. 5, in an LB medium with added ampicillin;
induction of the fusion protein synthesis by adding isopropylthiogalactoside;
lysis of the bacteria;
purification of the fusion protein obtained by chromatography on amylose-agarose resin and recovery of the fractions containing the purified protein.

8. Use of a protein according to claim 1 in a method for identifying modulators of the Cdc25B1, Cdc25B2, Cdc25B3 or Cdc25C protein, characterized in that said method comprises the following successive stages:
addition, of the fusion protein such as obtained by a process for the preparation according to claim 7 and of a compound presumed be a modulator of the Cdc25B1, Cdc25B2, Cdc25B3 or Cdc25C protein to a solution of 3-O-methylfluorescein phosphate;
determination of the quantity of 3-O-methylfluorescein produced in relation to the initial quantity of 3-O-methylfluorescein phosphate.

9. Use according to claim 8, characterized in that the determination of the quantity of 3-O-methylfluorescein produced in relation to the initial quantity of 3-O-methylfluorescein phosphate is carried out by measuring the absorbance linked to 3-O-methylfluoresceine at the wavelength of 477 nm.

10. Use according to claim 8, characterized in that the determination of the quantity of 3-O-methylfluoresceine produced in relation to the initial quantity of 3-O-methylfluoresceine phosphate is carried out by fluorometry using excitation at the wavelength of 475 nm and reading at the wavelength of 510 nm.


SEQUENCE LISTING (14 sequences)

SEQ ID NO: 1 - DNA, 2607 bp, Artificial Sequence
  Description of Artificial Sequence Synthetic MBP-Cdc25C fusion nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatcctctagaatgtctacggaactcttctcatccacaagagaggaaggaagctctggctcaggacccagttttaggtctaatcaaaggaaaatgttaaacctgctcctggagagagacacttcctttaccgtctgtccagatgtccctagaactccagtgggcaaatttcttggtgattctgcaaacctaagcattttgtctggaggaaccccaaaatgttgcctcgatctttcgaatcttagcagtggggagataactgccactcagcttaccacttctgcagaccttgatgaaactggtcacctggattcttcaggacttcaggaagtgcatttagctgggatgaatcatgaccagcacctaatgaaatgtagcccagcacagcttctttgtagcactccgaatggtttggaccgtggccatagaaagagagatgcaatgtgtagttcatctgcaaataaagaaaatgacaatggaaacttggtggacagtgaaatgaaatatttgggcagtcccattactactgttccaaaattggataaaaatccaaacctaggagaagaccaggcagaagagatttcagatgaattaatggagttttccctgaaagatcaagaagcaaaggtgagcagaagtggcctatatcgctccccgtcgatgccagagaacttgaacaggccaagactgaagcaggtggaaaaattcaaggacaacacaataccagataaagttaaaaaaaagtatttttctggccaaggaaagctcaggaagggcttatgtttaaagaagacagtctctctgtgtgacattactatcactcagatgctggaggaagattctaaccaggggcacctgattggtgatttttccaaggtatgtgcgctgccaaccgtgtcagggaaacaccaagatctgaagtatgtcaacccagaaacagtggctgccttactgtcggggaagttccagggtctgattgagaagttttatgtcattgattgtcgctatccatatgagtatctgggaggacacatccagggagccttaaacttatatagtcaggaagaactgtttaacttctttctgaagaagcccatcgtccctttggacacccagaagagaataatcatcgtgttccactgtgaattctcctcagagaggggcccccgaatgtgccgctgtctgcgtgaagaggacaggtctctgaaccagtatcctgcattgtactacccagagctatatatccttaaaggcggctacagagacttctttccagaatatatggaactgtgtgaaccacagagctactgccctatgcatcatcaggaccacaagactgagttgctgaggtgtcgaagccagagcaaagtgcaggaaggggagcggcagctgcgggagcagattgcccttctggtgaaggacatgagcccatga

SEQ ID NO: 2 - DNA, 1461 bp, Artificial Sequence
  Description of Artificial Sequence Synthetic Hs Cdc25C nucleic acid sequence with its XbaI restriction ends
tctagaatgtctacggaactcttctcatccacaagagaggaaggaagctctggctcaggacccagttttaggtctaatcaaaggaaaatgttaaacctgctcctggagagagacacttcctttaccgtctgtccagatgtccctagaactccagtgggcaaatttcttggtgattctgcaaacctaagcattttgtctggaggaaccccaaaatgttgcctcgatctttcgaatcttagcagtggggagataactgccactcagcttaccacttctgcagaccttgatgaaactggtcacctggattcttcaggacttcaggaagtgcatttagctgggatgaatcatgaccagcacctaatgaaatgtagcccagcacagcttctttgtagcactccgaatggtttggaccgtggccatagaaagagagatgcaatgtgtagttcatctgcaaataaagaaaatgacaatggaaacttggtggacagtgaaatgaaatatttgggcagtcccattactactgttccaaaattggataaaaatccaaacctaggagaagaccaggcagaagagatttcagatgaattaatggagttttccctgaaagatcaagaagcaaaggtgagcagaagtggcctatatcgctccccgtcgatgccagagaacttgaacaggccaagactgaagcaggtggaaaaattcaaggacaacacaataccagataaagttaaaaaaaagtatttttctggccaaggaaagctcaggaagggcttatgtttaaagaagacagtctctctgtgtgacattactatcactcagatgctggaggaagattctaaccaggggcacctgattggtgatttttccaaggtatgtgcgctgccaaccgtgtcagggaaacaccaagatctgaagtatgtcaacccagaaacagtggctgccttactgtcggggaagttccagggtctgattgagaagttttatgtcattgattgtcgctatccatatgagtatctgggaggacacatccagggagccttaaacttatatagtcaggaagaactgtttaacttctttctgaagaagcccatcgtccctttggacacccagaagagaataatcatcgtgttccactgtgaattctcctcagagaggggcccccgaatgtgccgctgtctgcgtgaagaggacaggtctctgaaccagtatcctgcattgtactacccagagctatatatccttaaaggcggctacagagacttctttccagaatatatggaactgtgtgaaccacagagctactgccctatgcatcatcaggaccacaagactgagttgctgaggtgtcgaagccagagcaaagtgcaggaaggggagcggcagctgcgggagcagattgcccttctggtgaaggacatgagcccatgataacattccagccactggctgctaacatctaga

SEQ ID NO: 3 - DNA, 24 bp, Artificial Sequence
  Description of Artificial Sequence XbaI SENSE primer
gttctagaatgtctagaactcttc

SEQ ID NO: 4 - DNA, 18 bp, Artificial Sequence
  Description of Artificial Sequence XbaI ANTI- SENSE primer
ggctctgagttgcgccgg

SEQ ID NO: 5 - DNA, 8101 bp, Artificial Sequence
  Description of Artificial Sequence Synthetic JM109 / pMAL - Hs Cdc25C strain
ccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcacttcaccaacaaggaccatagattatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatcctctagaatgtctacggaactcttctcatccacaagagaggaaggaagctctggctcaggacccagttttaggtctaatcaaaggaaaatgttaaacctgctcctggagagagacacttcctttaccgtctgtccagatgtccctagaactccagtgggcaaatttcttggtgattctgcaaacctaagcattttgtctggaggaaccccaaaatgttgcctcgatctttcgaatcttagcagtggggagataactgccactcagcttaccacttctgcagaccttgatgaaactggtcacctggattcttcaggacttcaggaagtgcatttagctgggatgaatcatgaccagcacctaatgaaatgtagcccagcacagcttctttgtagcactccgaatggtttggaccgtggccatagaaagagagatgcaatgtgtagttcatctgcaaataaagaaaatgacaatggaaacttggtggacagtgaaatgaaatatttgggcagtcccattactactgttccaaaattggataaaaatccaaacctaggagaagaccaggcagaagagatttcagatgaattaatggagttttccctgaaagatcaagaagcaaaggtgagcagaagtggcctatatcgctccccgtcgatgccagagaacttgaacaggccaagactgaagcaggtggaaaaattcaaggacaacacaataccagataaagttaaaaaaaagtatttttctggccaaggaaagctcaggaagggcttatgtttaaagaagacagtctctctgtgtgacattactatcactcagatgctggaggaagattctaaccaggggcacctgattggtgatttttccaaggtatgtgcgctgccaaccgtgtcagggaaacaccaagatctgaagtatgtcaacccagaaacagtggctgccttactgtcggggaagttccagggtctgattgagaagttttatgtcattgattgtcgctatccatatgagtatctgggaggacacatccagggagccttaaacttatatagtcaggaagaactgtttaacttctttctgaagaagcccatcgtccctttggacacccagaagagaataatcatcgtgttccactgtgaattctcctcagagaggggcccccgaatgtgccgctgtctgcgtgaagaggacaggtctctgaaccagtatcctgcattgtactacccagagctatatatccttaaaggcggctacagagacttctttccagaatatatggaactgtgtgaaccacagagctactgccctatgcatcatcaggaccacaagactgagttgctgaggtgtcgaagccagagcaaagtgcaggaaggggagcggcagctgcgggagcagattgcccttctggtgaaggacatgagcccatgataacattccagccactggctgctaacatctagagtcgacctgcaggcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcagcttggctgttttggcggatgagataagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 6 - DNA, 2063 bp, Artificial Sequence
  Description of Artificial Sequence Synthetic Hs Cdc25B1 nucleic acid sequence with its NdeI and BamH1 restriction ends
catatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 7 - DNA, 1982 bp, Artificial Sequence
  Description of Artificial Sequence Synthetic Hs Cdc25B2 nucleic acid sequence with its NdeI and BamH1 restriction ends
catatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 8 - DNA, 2105 bp, Artificial Sequence
  Description of Artificial Sequence Synthetic Hs Cdc25B3 nucleic acid sequence with its NdeI and BamH1 restriction ends
catatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 9 - DNA, 3236 bp, Artificial Sequence
  Description of Artificial Sequence Synthetic JM109 / pMAL - Hs Cdc25B1 strain
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 10 - DNA, 4955 bp, Artificial Sequence
  Description of Artificial Sequence Synthetic JM109 / pMAL - Hs Cdc25B2 strain
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaaatgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 11 - DNA, 3278 bp, Artificial Sequence
  Description of Artificial Sequence Synthetic JM109 / pMAL - Hs Cdc25B3 strain
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggatcc

SEQ ID NO: 12 - DNA, 2877 bp, Artificial Sequence
  Description of Artificial Sequence Synthetic MBP-Cdc25B1 fusion nucleic acid sequence
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtga

SEQ ID NO: 13 - DNA, 2796 bp, Artificial Sequence
  Description of Artificial Sequence Synthetic MBP-Cdc25B2 fusion nucleic acid sequence
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtga

SEQ ID NO: 14 - DNA, 2919 bp, Artificial Sequence
  Description of Artificial Sequence Synthetic MBP-Cdc25B3 fusion nucleic acid sequence
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattccatatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtga